(19) United States
(12) Patent Application Publication
(10) Pub. No.: US 2007/0032535 A1
(43) Pub. Date: Feb. 8, 2007

(54) Diastereomeric dynamic kinetic resolution process for preparing (+)-(2s,3s)-2-(3-chlorophenyl)-3,5,5-trimethyl-2-morpholinol, salts, and solvates thereof

(75) Inventor: John Joseph Partridge, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: Smith Kline Beecham Corporation D/b/a Glaxo Smith Kline, Research Triangle Park, NC (US)

(21) Appl. No.: 10/577,172

(22) PCT Filed: Oct. 21, 2004
(86) PCT No.: PCT/US04/34667

§ 371 Date: Apr. 26, 2006

(51) Int. Cl. A61K 31/42; A01N 43/80

(52) U.S. Cl. 514/378

(57) ABSTRACT

Disclosed is a method for preparing (+)-(2S,3S)-2-(3-chlorophenyl)-3,5,5-trimethyl-2-morpholinol, pharmaceutically acceptable salts, and pharmaceutically acceptable solvates thereof, such as the (+)-(2S,3S)-2-(3-chlorophenyl)-3,5,5-trimethyl-2-morpholinol hydrochloride salt via dynamic kinetic resolutions.

### BACKGROUND OF THE INVENTION

1. Field of the Invention

The present invention relates to a process for making (+)-(2S,3S)-2-(3-chlorophenyl)-3,5,5-trimethyl-2-morpholinol, pharmaceutically acceptable salts, and pharmaceutically acceptable solvates thereof such as the (+)-(2S,3S)-2-(3-chlorophenyl)-3,5,5-trimethyl-2-morpholinol hydrochloride salt by a dynamic kinetic resolution of the racemate (±)-(2R*,3R*)-2-(3-chlorophenyl)-3,5,5-trimethyl-2-morpholinol.

2. Description of the Prior Art

(+)-(2S,3S)-2-(3-chlorophenyl)-3,5,5-trimethyl-2-morpholinol and pharmaceutically acceptable salts thereof, and pharmaceutically acceptable solvates thereof, and pharmaceutical compositions comprising the same are used in treating numerous diseases or disorders such as depression, attention deficit hyperactivity disorder (ADHD), obesity, migraine, pain, sexual dysfunction, Parkinson's disease, Alzheimer's disease, seasonal affective disorder (SAD), addiction to cocaine, addiction to alcohol, and addiction to nicotine-containing products (especially tobacco).

Several literature references describe the preparation of either the (+)-(2S,3S)— or (—)-(2R,3R)-enantiomers from (±)-(2R*,3R*)-2-(3-chlorophenyl)-3,5,5-trimethyl-2-morpholinol. For instance, reference is made to U.S. Pat. No. 6,342,496 B1, issued to Jerussi et al. on Jan. 29, 2002, U.S. Pat. No. 6,337,328 B1, issued to Fang et al. on Jan. 8, 2002, U.S. Published Applications 2002/0052340 A1, and 2002/0052341 A1, as well as WO 01/62257 A2. Reference is also made to pending U.S. application Ser. No. 10/147,588. Also of interest are U.S. Pat. Nos. 6,274,579; 6,391,875; and 6,734,213.

U.S. Pat. No. 6,337,328, U.S. Published Application 2002/0052341 A1, WO 01/62257 A2, and U.S. application Ser. No. 10/147,588 refer to a chiral acid resolution method for preparing (+)-(2S,3S)-2-(3-chlorophenyl)-3,5,5-trimethyl-2-morpholinol from the racemate (±)-(2R*,3R*)-2-(3-chlorophenyl)-3,5,5-trimethyl-2-morpholinol. However, the method described in each of these references differs from the present invention in both procedure and result. These references relate to chemical resolutions of the racemate, while the present invention involves the dynamic kinetic resolution of the racemate. In the simple chemical resolution of the racemate, these references must isolate the desired diastereomeric morpholinol from a mixture of diastereomeric salts. The maximum yield of the desired diastereomer can therefore be at most about 50%.

In general, most chemical resolutions of a racemic material, such as Fang et al and Jerussi et al, produce the desired enantiomer or mirror image diastereoisomer in a maximum theoretical yield of 50%. Generally, the undesired enantiomer or mirror image diastereoisomer is discarded as waste in the mother liquor.

There are a few instances in which a maximum theoretical yield of 100% of a specific enantiomer can be obtained by a chiral enzymatic reaction on a pro-chiral substrate. This process is sometimes termed “an enzymatic hydrolytic desymmetrization”. Such a chiral enzymatic reaction on a pro-chiral substrate for five closely related compounds is set forth in “Enantioselective Hydrolysis of cis-3,5-Diacetoxycyclopentene: 1R,4S-(+)-4-Hydroxy-2-cyclopentenyl Acetate”, Deardorff, D. R., Windham, C. Q. and Craney, C. L., Org. Synth. Coll. Vol. IX, 1998, 487-493 for five closely related compounds.

Likewise, there are few instances in which a dynamic kinetic resolution can be employed to give a maximum theoretical yield of 100% of a desired specified enantiomer, via equilibration of the enantiomers during the resolution. An example of this rare type of chemical dynamic kinetic resolution can be found in Reider, P. J., Davis, P., Hughes, D. L. and Grabowski, E. J. J., J. Org. Chem., 1987, 52, 955. An example of a rare alpha-substituted ketone reductive dynamic kinetic resolution leading largely to a single diastereoisomer is described in Yamada, S., Mori, Y., Morimatsu, K., Ishizu, Y., Ozaki, Y., Yoshioka, R., Nakatani, T., and Seko, H., J. Org. Chem., 1996, 61, 8586.

In general, true diastereoisomeric dynamic kinetic resolutions are extremely rare for the preparation of a single, pure diastereoisomer (a compound containing two chiral centers), since both chiral centers must be capable of equilibration. In this special case of diastereoisomer dynamic kinetic resolutions, only one of four possible chiral diastereoisomers is formed.

### SUMMARY OF THE INVENTION

There exists a need for a reaction to produce (+)-(2S,3S)-2-(3-chlorophenyl)-3,5,5-trimethyl-2-morpholinol from its two chiral center-containing racemate in a greater than about 50% yield as is typical in a simple chemical isolation process. There especially exists the need to produce this compound in a yield approximating 100%, that is, greater than about 60% yield, preferably greater than about 75% yield. Therefore, according to the present invention, there is provided a dynamic kinetic resolution method of preparing the compound (+)-(2S,3S)-2-(3-chlorophenyl)-3,5,5-trimethyl-2-morpholinol and/or desired salt or solvate forms from the corresponding racemate, which racemate will be referred to herein as (±)-(2R*,3R*)-2-(3-chlorophenyl)-3,5,5-trimethyl-2-morpholinol.

It is believed that when the present invention is compared with prior methods of isolation (e.g. a simple chemical isolation or resolution), it will be apparent that according to the present invention, there will be a much higher yield (greater than about 50% yield and generally greater than about 80% yield). Further, probably there will be no need to isolate a chiral salt of (+)-(2S,3S)-2-(3-chlorophenyl)-3,5,5-trimethyl-2-morpholinol from the mixture of diastereomeric salts. Additionally, it is believed that there will be little or no mother liquor left over for disposal as waste.

The present invention achieves one or more of these desirable results by performing a diastereoisomeric dynamic kinetic resolution at mildly basic to basic pH, namely about pH 8 to about pH 12 as illustrated in the schematic diagram herein. As can be seen in the schematic diagram, this allows the undesired 2R,3R-morpholinol to “unravel” to the undesired 2R-hydroxybupropion, then to be racemized or equilibrated with the desired 2S-hydroxybupropion, which then “ravels” back up to the desired 2S,3S -morpholinol which precipitates out as a solid acid salt in amorphous or preferably crystalline form. This method will produce a desired chiral acid salt in a greater than about 60% yield, preferably in about an 80% to about 100% yield, most preferably about 90% to about 100% yield, preferably with little or no mother liquor left over. This can be beneficial to the environment and/or eliminates further processing of the mother liquor before disposing of it.

That is, the present invention provides a process for preparing (+)-(2S,3S)-2-(3-chlorophenyl)-3,5,5-trimethyl-2-morpholinol in its free base, its salt form, or both, which process comprises a dynamic kinetic resolution by equilibrating the two chiral centers of (±)-(2R*,3R*)-2-(3-chlorophenyl)-3,5,5-trimethyl-2-morpholinol.

To summarize, the present invention provides a process for making (+)-(2S,3S)-2-(3-chlorophenyl)-3,5,5-trimethyl-2-morpholinol which process comprises:

(1) treating (±)-(2R*,3R*)-2-(3-chlorophenyl)-3,5,5-trimethyl-2-morpholinol dissolved in a solvent with at least one base to give a solution having a pH of about pH 8 to about pH 12;

(2) adding at least 0.5 equivalent of a chiral acid (preferably slowly) with stirring while maintaining the pH of the solution between about pH 8 to about pH 12 by addition of additional base;

(3) adding seed crystals of the desired chiral acid salt of (+)-(2S,3S)-2-(3-chlorophenyl)-3,5,5-trimethyl-2-morpholinol with stirring; and

(4) adding (preferably slowly) at least 0.5 equivalent of additional chiral acid at pH 8 to pH 12 (with additional base added as needed) and then adjusting the pH of the solution with acid until the pH of the solution reaches about pH 6 to about pH 8.

In a preferred embodiment, the present invention provides a process for making (+)-(2S,3S)-2-(3-chlorophenyl)-3,5,5-trimethyl-2-morpholinol which process comprises:

(1) treating (±)-(2R*,3R*)-2-(3-chlorophenyl)-3,5,5-trimethyl-2-morpholinol dissolved in a solvent with at least one base to give a solution having a pH of about pH 8 to about pH 12;

(2) adding (preferably slowly) at least 0.5 equivalent of a chiral acid with stirring while maintaining the pH of the solution between about pH 8 to about pH 12 by addition of additional base;

(3) adding seed crystals of the desired chiral acid salt of (+)-(2S,3S)-2-(3-chlorophenyl)-3,5,5-trimethyl-2-morpholinol with stirring;

(4) adding (preferably slowly) at least 0.5 equivalent of additional chiral acid at pH 8 to pH 12 (with additional base added as needed) and then adjusting the pH of the solution with acid until the pH reaches about pH 6 to about pH 8;

(5) isolating a precipitated solid (crystalline or amorphous) acid salt of (+)-(2S,3S)-2-(3-chlorophenyl)-3,5,5-trimethyl-2-morpholinol;

(6) converting said solid (crystalline or amorphous) acid salt of (+)-(2S,3S)-2-(3-chlorophenyl)-3,5,5-trimethyl-2-morpholinol into (+)-(2S,3S)-2-(3-chlorophenyl)-3,5,5-trimethyl-2-morpholinol free base;

(7) converting the (+)-(2S,3S)-2-(3-chlorophenyl)-3,5,5-trimethyl-2-morpholinol free base into (+)-(2S,3S)-2-(3-chlorophenyl)-3,5,5-trimethyl-2-morpholinol salt; and.

(8) recrystallizing said salt of step 7 to produce a purer form of the (+)-(2S,3S)-2-(3-chlorophenyl)-3,5,5-trimethyl-2-morpholinol salt.

The schematic diagram below details the present dynamic kinetic resolution method for morpholinols.

### Dynamic Kinetic Resolution of Morpholinols

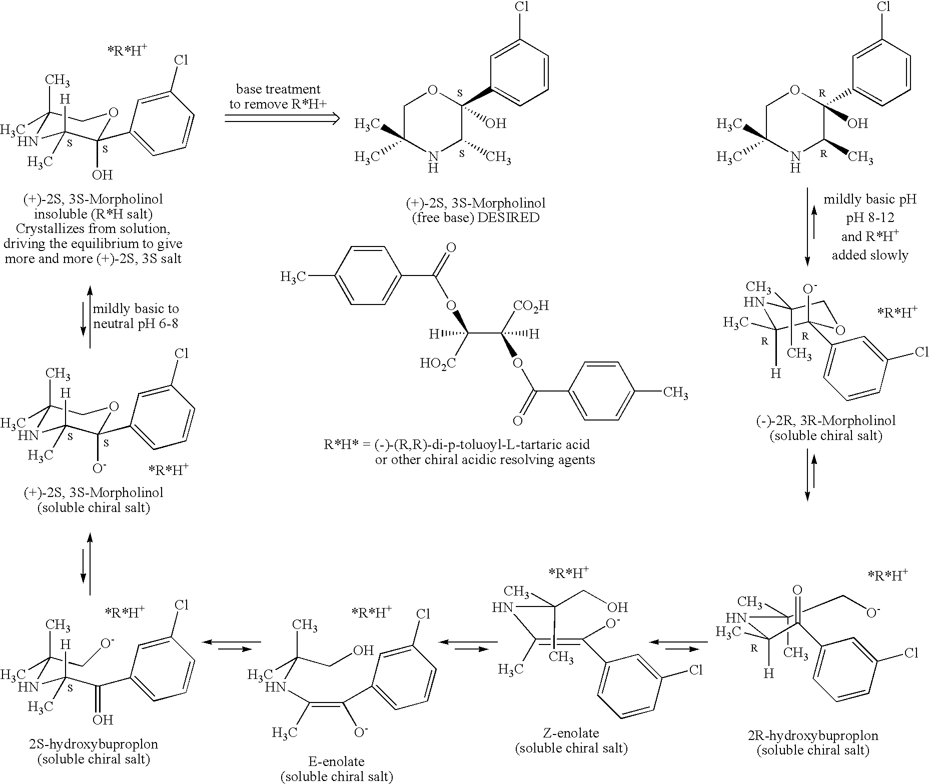

In a further embodiment of the invention there is provided a pharmaceutical composition comprising an active ingredient of (+)-(2S,3S)-2-(3-chlorophenyl)-3,5,5-trimethyl-2-morpholinol, a pharmaceutically acceptable salt thereof, or a pharmaceutically acceptable solvate thereof prepared in accordance with the process described herein together with at least one pharmaceutically acceptable excipient.

In still another embodiment of the invention there is provided a method of treatment comprising the administration (preferably oral) to a mammal (preferably a human) of an active ingredient comprising (+)-(2S,3S)-2-(3-chlorophenyl)-3,5,5-trimethyl-2-morpholinol, a pharmaceutically acceptable salt thereof, or a pharmaceutically acceptable solvate thereof prepared in accordance with the process described herein together with at least one pharmaceutically acceptable excipient.

In another embodiment there is provided the use of (+)-(2S,3S)-2-(3-chlorophenyl)-3,5,5-trimethyl-2-morpholinol, a pharmaceutically acceptable salt thereof, or pharmaceutically acceptable solvate thereof prepared in accordance with the process described herein in the manufacture of a medicament. Such medicament can be used for the treatment of depression (major and bipolar), attention deficit hyperactivity disorder (ADHD), anxiety, obesity, migraine, pain, sexual dysfunction in both men and women, Parkinson's disease, Alzheimer's disease, seasonal affective disorder (SAD), addiction to alcohol, addiction to cocaine, and addiction to nicotine-containing products (e.g., tobacco).

### DETAILED DESCRIPTION OF THE INVENTION

### 1. Introduction

The present invention provides a method for making (+)-(2S,3S)-2-(3-chlorophenyl)-3,5,5-trimethyl-2-morpholinol, a single diastereoisomer from a two-chiral center racemate. The process is an example of a crystallization-induced asymmetric transformation, also termed “a second-order asymmetric transformation”, but with the important distinction that there are two chiral centers equilibrating. (For one chiral center equilibrating asymmetric transformations see “Crystallization-Induced Asymmetric Transformations” in Jacques, J., Collet, A. and Wilen, S. H., in Enantiomers, Racemates and Resolutions, Krieger Publishing Company, Malabar, Fla., 1991, Chapter 6, pp. 369-377). These processes are also referred to as a dynamic kinetic resolutions as disclosed in “Enantioselective Synthesis: The Optimum Solution”, Partridge, J. J. and Bray, B. L. in Process Chemistry in the Pharmaceutical Industry, (Gadamasetti, K. G., Ed.) Marcel Dekker, New York, N.Y., 1999, pp. 314-315.

In the process of the invention the following steps are performed:

(1) treating (±)-(2R*,3R*)-2-(3-chlorophenyl)-3,5,5-trimethyl-2-morpholinol dissolved in a solvent with at least one base to give a solution or mixture having a pH of about pH 8 to about pH 12;

(2) adding at least 0.5 equivalent of a chiral acid (preferably slowly) with stirring while maintaining the pH of the solution or mixture between about pH 8 to about pH 12 by addition of additional base;

(3) adding seed crystals of the desired acid salt of (+)-(2S,3S)-2-(3-chlorophenyl)-3,5,5-trimethyl-2-morpholinol with stirring; and

(4) adding (preferably slowly) at least 0.5 equivalent of additional chiral acid at a pH 8 to pH 12 (with additional base added as needed) and then adjusting the pH of the mixture or solution with acid until the pH of the solution or mixture reaches about pH 6 to about pH 8.

The following additional steps are performed in a preferred embodiment:

(5) isolating a precipitated solid (amorphous or crystalline) acid salt of (+)-(2S,3S)-2-(3-chlorophenyl)-3,5,5-trimethyl-2-morpholinol;

(6) converting said precipitated solid (amorphous or crystalline) acid salt of (+)-(2S,3S)-2-(3-chlorophenyl)-3,5,5-trimethyl-2-morpholinol into (+)-(2S,3S)-2-(3-chlorophenyl)-3,5,5-trimethyl-2-morpholinol free base;

(7) converting the (+)-(2S,3S)-2-(3-chlorophenyl)-3,5,5-trimethyl-2-morpholinol free base into (+)-(2S,3S)-2-(3-chlorophenyl)-3,5,5-trimethyl-2-morpholinol salt; and

(8) recrystallizing said salt of step 7 to produce a purer form of the (+)-(2S,3S)-2-(3-chlorophenyl)-3,5,5-trimethyl-2-morpholinol salt.

The objective of the above-described process is to produce 99%+ enantiomeric excess (99% ee) pure (+)-(2S,3S)-2-(3-chlorophenyl)-3,5,5-trimethyl-2-morpholinol salt.

### 2. Diastereomeric Dynamic Kinetic Resolution Process to Prepare (+)-(2S,3S)-2-(3-chlorophenyl)-3,5,5-trimethyl-2-morpholinol and Pharmaceutically Acceptable Salts

Step One: Treating (±)-(2R*,3R*)-2-(3-chlorophenyl)-3,5,5-trimethyl-2-morpholinol dissolved in a solvent with at least one base to give a solution or mixture having a pH of about pH 8 to about pH 12.

In this step, (±)-(2R*,3R*)-2-(3-chlorophenyl)-3,5,5-trimethyl-2-morpholinol is dissolved in a solvent.

Suitable solvents include protic solvents such as methanol and ethanol, ketones such as acetone and mixed solvents such as water-acetonitrile, methanol-acetonitrile, water-acetone, water-dimethylformamide, and the like. Typical concentrations of the racemate morpholinol in a given solvent or solvent combination are about 0.01 molar to about 2.0 molar. The type and amount of solvent should be selected so as to completely or substantially (that is, over 30% dissolution) dissolve the (±)-(2R*,3R*)-2-(3-chlorophenyl)-3,5,5-trimethyl-2-morpholinol. By choosing the desired solvent(s) and adjusting the amount of solvent(s) employed using routine experimentation or other known methods, the dynamic kinetic resolution will be allowed to take place efficiently leading to higher yields of the desired chiral end-product: (+)-(2S,3S)-2-(3-chlorophenyl)-3,5,5-trimethyl-2-morpholinol.

The dissolved (±)-(2R*,3R*)-2-(3-chlorophenyl)-3,5,5-trimethyl-2-morpholinol is treated with a base. The type and amount of base added should be selected so as to result in a solution with a pH between about pH 8 to about pH 12. The basic pH between about pH 8 to about pH 12 allows the undesired 2R,3R-morpholinol to “unravel” to the undesired 2R-hydroxybupropion, then to be racemized or equilibrated with the desired 2S-hydroxybupropion which then “ravels” back up to the desired 2S,3S -morpholinol which will crystallize or precipitate out as a solid acid salt. This may allow production of the desired chiral acid salt in about an 80-100% yield, with little or no mother liquor left over.

Suitable bases include, but are not limited to tertiary amine bases such as trimethylamine and triethylamine; aromatic bases such as pyridine; inorganic bases such as ammonium hydroxide, sodium bicarbonate, sodium carbonate, sodium hydroxide, potassium bicarbonate, potassium carbonate and potassium hydroxide. The dissolved racemate to base ratio would be about 10:1 to about 1:1.

A solution or mixture with a pH between about pH 8 to about pH 12 results. A pH range of about pH 9 to pH 11 is preferred.

Step Two: Adding at least 0.5 equivalent of a chiral acid (preferably slowly) with stirring at about 30 to about 300 revolutions per minute (rpms) while maintaining the pH of the solution between about pH 8 to about pH 12 by addition of additional base.

It can be seen that step 2 and step 4 together require a total of at least 1.0 equivalent. Up to about 2.0 equivalents of chiral acid can be employed in steps 2 and 4 taken together. The type and amount of chiral acid should be selected so as to form chiral acid salts with all available desired enantiomers. The addition of too little chiral acid (less than about 1 equivalent) will result in lower yields of desired chiral acid salt; and therefore lower yields of the desired chiral end-product: (+)-(2S,3S)-2-(3-chlorophenyl)-3,5,5-trimethyl-2-morpholinol. The addition of more than 1 equivalent of chiral acid should not impair the desired high yields.

Suitable chiral acids include, but are not limited to, L-tartaric acid, D-tartaric acid, (−)-(R,R)-di-p-benzoyl-L-tartaric acid, (+)-(S,S)-di-p-benzoyl-D-tartaric acid, (−)-(R,R)-di-p-toluoyl-L-tartaric acid, (+)-(S,S)-di-p-toluoyl-D-tartaric acid, (−)-(1R)-10-camphorsulfonic acid, (+)-(1S)-10-camphorsulfonic acid, D- or L-malic acid, D- or L-mandelic acid and the like.

The chiral acid is added, preferably slowly while stirring. Preferably, the chiral acid is added slowly by adding the chiral acid in a solid form, in a suspension, or in a solution portionwise (in aliquots) or at a rate of about 1 gram to 100 grams of chiral acid per minute. If the stirring is too fast, crystallization may not be uniform and/or the stirring shaft may break or the stirring shaft motor may overheat. If the stirring is too slow, crystallization may not be uniform or may occur too quickly leading to a large amount of solid in a ball. This is undesirable and may also cause the stirring shaft to break or the stirring shaft motor to overheat. Stirring is at a rate of about 30 to about 300 revolutions per minute (about 30 to about 300 rpms).

The stirring may be provided by any means, for instance, by mechanical stirring with one of a number of types of commercially available paddle stirrers (e.g., a sintered glass filter funnel, a Gooch filter, a pan filter, and a Rosemund filter).

In this step, the pH is maintained between about pH 8 to about pH 12. A pH range of about pH 9 to about pH 11 is preferred. Again, the pH is important in performing the present diastereoisomeric dynamic kinetic resolution. The basic pH, namely, from about pH 8 to about pH 12 allows the undesired 2R,3R-morpholinol to “unravel” to the undesired 2R-hydroxybupropion, then to be racemized or equilibrated with the desired 2S-hydroxybupropion which then “ravels” back up to the desired 2S,3S -morpholinol which crystallizes or precipitates out as a solid acid salt.

If the pH is too high, (about pH 12 to about 14), this may saponify the resolving agent. For instance, the p-toluolyl esters that are part of the most desired resolving agent (−)-(R,R)-di-p-toluoyl-L-tartaric acid may saponify to give degradants—the basic salts of p-toluic acid (i.e., p-toluic acid sodium salt as an example) and L-tartaric acid (i.e., L-tartaric acid, mono- or disodium salts as examples). Too high a pH may also degrade the (+)-(2S,3S)-2-(3-chlorophenyl)-3,5,5-trimethyl-2-morpholinol free base to a number of by-products, including the degradant—the basic salt of meta-chlorobenzoic acid (i.e. the meta-chlorobenzoic acid sodium salt as an example).

If the pH is too low, (pH less than about 5-7), there may be no equilibration of the (−)-(2R,3R)-2-(3-chlorophenyl)-3,5,5-trimethyl-2-morpholinol to the (+)-(2S,3S)-2-(3-chlorophenyl)-3,5,5-trimethyl-2-morpholinol via the 2R- and 2S-hydroxybupropion molecules (see schematic diagram above).

Additional base may be added to maintain this pH. Any of the above-listed bases are suitable. However, preferably the same base is used throughout the present dynamic kinetic resolution process. Typically, additional base is added slowly by adding the base in a solid or liquid form, in a suspension or in a solution dropwise or at a rate of about 0.5 grams per minute to about 50 grams per minute.

Step Three: Adding seed crystals of the desired acid salt of (+)-(2S,3S)-2-(3-chlorophenyl)-3,5,5-trimethyl-2-morpholinol with stirring.

In this step, seed crystals of the chiral acid salt of (+)-(2S,3S)-2-(3-chlorophenyl)-3,5,5-trimethyl-2-morpholinol are added with stirring. The seed crystals are added to provide a small amount of desired salt surface area to induce or provoke more of the desired salt to crystallize out. Since the (+)-(2S,3S)-2-(3-chlorophenyl)-3,5,5-trimethyl-2-morpholinol is desired, one would “seed” with a chiral salt of (+)-(2S,3S)-2-(3-chlorophenyl)-3,5,5-trimethyl-2-morpholinol. Thus, according to the present process, one can “drive” the resolution to produce up to nearly 100% of the desired acid salt of (+)-(2S,3S)-2-(3-chlorophenyl)-3,5,5-trimethyl-2-morpholinol. The seed crystals are obtained by reacting a pure sample of (+)-(2S,3S)-2-(3-chlorophenyl)-3,5,5-trimethyl-2-morpholinol with an equimolar amount of the desired acid such as (−)-(R,R)-di-p-toluoyl-L-tartaric acid.

The amount of seed crystals added includes, but is not limited to about 10 mg (for small lab scale reactions of about 10 mL total reaction volume) to about 1.0 gram (for large scale reactions of about a liter to multi-liter total reaction volumes) of (+)-(2S,3S)-2-(3-chlorophenyl)-3,5,5-trimethyl-2-morpholinol (−)-(R,R)-di-p-toluoyl-L-tartaric acid salt as a preferred example. If not enough seed crystals are added, crystallization will not proceed. The addition of too much seed crystals may cause the desired crystallization to proceed at a faster rate since more crystal surface area will be exposed to the reaction mixture which may lead to the same stirring problems as discussed above. Determination of the amount of seed crystals to be employed for a given reaction is a well known procedure for one skilled in the art of seed crystal induced crystallizations.

Selective crystallization begins during Step Three (3) and ends after Step Four (4) begins. In this selective crystallization, the acid salt precipitates or crystallizes out.

The stirring performed in this step may be done with mechanical stirring, for instance, with any of a number of types of commercially available paddle stirrers, at a stirring rate of at about 30 to about 300 revolutions per minute (rpms), preferably about 50 rpms to about 250 rpms, and most preferably about 75 rpms to about 200 rpms. If the stirring is too fast or too slow, the same problems discussed above may arise.

Step Four: Slowly adding at least 0.5 equivalent of additional chiral acid at a pH 8 to pH12 (with additional base added as needed) and then adjusting the pH of the mixture or solution with acid until the pH of the solution or mixture reaches about pH 6 to about pH 8.

In this step, at least 0.5 equivalent of additional chiral acid (with the addition of the same chiral acid selected in previous steps being preferred) is added at pH 8 to pH 12 with additional base added as needed. Then the pH of the mixture or solution is adjusted with chiral acid to reach about pH 6 to about pH8. The additional chiral acid is preferably added slowly, preferably within about 1 to 4 hours to produce an amorphous, a crystalline, or a precipitated acid salt of (+)-(2S,3S)-2-(3-chlorophenyl)-3,5,5-trimethyl-2-morpholinol. The slow addition is conducted as set forth previously in Step 2.

With the addition of the additional chiral acid, the reaction mixture is titrated to a pH of about pH 6 to about pH 8. A preferred pH range is about pH 6.5 to about pH 7.5. The purpose of this titration is to bring the reaction mixture to a “neutral” pH (i.e., close to pH 7). This final “neutralization” to pH 7 can also be carried out with a relatively inexpensive achiral acid, such as hydrochloric acid, or sulfuric acid.

Step Five: Isolation of a solid acid salt of (+)-(2S,3S)-2-(3-chlorophenyl)-3,5,5-trimethyl-2-morpholinol.

In this step, the solids from Step Four (4), are isolated. The isolation may be performed by any suitable method known to those skilled in the art of isolation of solids. For instance, the isolation can be performed on a sintered glass filter funnel, a Gooch filter, a pan filter or a Rosemund filter. The isolation may also be performed by decantation of the liquid phase from the solid mass of desired product acid salt of (+)-(2S,3S)-2-(3-chlorophenyl)-3,5,5-trimethyl-2-morpholinol. Preferably, a filtration is used.

Step Six: Conversion of crystalline acid salt of (+)-(2S,3S)-2-(3-chlorophenyl)-3,5,5-trimethyl-2-morpholinol from Step Five (5) into (+)-(2S,3S)-2-(3-chlorophenyl)-3,5,5-trimethyl-2-morpholinol free base. An amine-containing compound is commonly termed an “amine free base” or “free base” if the amine exists in a non-protonated or non-salt form. In this step, the solid acid salt of the morpholinol is converted into its free base form. This may be accomplished by treating the acid salt with a base, preferably an excess of a strong base such as ammonium hydroxide, potassium hydroxide or sodium hydroxide in water to pH greater than about pH 10. The pH must be basic enough to convert the chiral acid resolving agent to its basic salt (i.e. ammonium salt, sodium salt), but not so basic (about pH 12 to about pH 14) that the chiral acid resolving agent is saponified, and the desired (+)-(2S,3S)-2-(3-chlorophenyl)-3,5,5-trimethyl-2-morpholinol free base is otherwise degraded as discussed above in Step 2. The type and amount of base should be selected so as to completely or substantially (over about 90%) convert all of the crystalline acid salt of (+)-(2S,3S)-2-(3-chlorophenyl)-3,5,5-trimethyl-2-morpholinol into its free base form.

The isolation of the free base from this reaction mixture is then accomplished by extraction of the free base into an organic solvent, e.g., methylene chloride, ethyl acetate, isopropyl acetate, methyl t-butyl ether and the like. The type and amount of organic solvent should be selected so as to completely, or substantially (over about 90%), extract the free base into the organic phase. If not enough organic solvent is used, not all of the free base will be extracted from the aqueous phase into the organic phase. If too much organic solvent is used, the final evaporation of the organic phase will take longer than necessary. Typically, the amount of organic solvent employed ranges from the amount needed to make about a 0.001 molar to about a 10 molar solution of (+)-(2S,3S)-2-(3-chlorophenyl)-3,5,5-trimethyl-2-morpholinol free base, preferably the amount needed to make about a 0.01 molar to about a 1.0 molar solution. The organic phase is then separated from the aqueous phase. Evaporation of the organic phase over about a 0.5 hour to about a 4.0 hour period then yields the desired (+)-(2S,3S)-2-(3-chlorophenyl)-3,5,5-trimethyl-2-morpholinol free base. (The aqueous phase contains the water-soluble salt of the chiral acid resolving agent.)

Step Seven: Conversion of (+)(2S,3S)-2-(3-chlorophenyl)-3,5,5-trimethyl-2-morpholinol free base from Step Six (6) into (+)-(2S,3S)-2-(3-chlorophenyl)-3,5,5-trimethyl-2-morpholinol hydrochloride salt.

This step may be performed by addition of more than one equivalent of hydrochloric acid or hydrogen chloride gas to the organic solvent solution of (+)-(2S,3S)-2-(3-chlorophenyl)-3,5,5-trimethyl-2-morpholinol free base from Step 6, to effect the formation of solid into (+)-(2S,3S)-2-(3-chlorophenyl)-3,5,5-trimethyl-2-morpholinol hydrochloride salt. This organic solvent solution is made from organic solvents such as methanol, ethanol, and acetonitrile. Additionally, a second solvent called an anti-solvent can also be added to more efficiently effect the formation of solid into (+)-(2S,3S)-2-(3-chlorophenyl)-3,5,5-trimethyl-2-morpholinol hydrochloride salt. The second solvent or anti-solvent should have less solvating properties than the original solvent toward into (+)-(2S,3S)-2-(3-chlorophenyl)-3,5,5-trimethyl-2-morpholinol hydrochloride salt. When more than one equivalent of hydrochloric acid or hydrogen chloride gas is added to the reaction mixture, the pH of the solution or mixture can reach a pH of about pH 1 to about pH 2. The amount of either hydrochloric acid or hydrogen chloride gas should be selected so as to completely, or substantially (over about 90%), convert the free base into the hydrochloride salt form. If not enough of either the hydrochloric acid or hydrochloride gas is used, the conversion will be incomplete and the yield will therefore be reduced. If too much of either the hydrochloric acid or hydrogen chloride is used, there should be no problem (other than excess waste generation).

Also, the type and amount of second organic solvent or anti-solvent should be selected to aid in the dissolving of the (+)-(2S,3S)-2-(3-chlorophenyl)-3,5,5-trimethyl-2-morpholinol free base or to aid in the precipitating of the desired final product (+)-(2S,3S)-2-(3-chlorophenyl)-3,5,5-trimethyl-2-morpholinol hydrochloride salt. Amounts and ranges of hydrogen chloride or hydrochloric acid and solvents are at least about 1 equivalent of hydrogen chloride in an organic solvent or about 1 equivalent of hydrochloric acid (i.e. hydrogen chloride in aqueous solvent). Suitable organic solvents include alcohols such as methanol and ethanol; aprotic polar solvents such as acetonitrile, dimethformamide, and the like. Suitable second organic solvents or anti-solvents can include esters such as ethyl acetate and isopropyl acetate; ethers such as diethyl ether, methyl tert-butyl ether, and diphenyl ether; aromatic hydrocarbons such as benzene and toluene; and aliphatic hydrocarbons such as hexanes and heptanes. In the examples (Examples 1d and 2d below), the (+)-(2S,3S)-2-(3-chlorophenyl)-3,5,5-trimethyl-2-morpholinol hydrochloride salt is dissolved in methanol, filtered and the anti-solvent ethyl acetate is added. Under vacuum, sufficient methanol is selectively removed so that the (+)-(2S,3S)-2-(3-chlorophenyl)-3,5,5-trimethyl-2-morpholinol hydrochloride salt crystallizes or precipitates from a solution comprising mainly ethyl acetate (probably about 50% to about 100% of the original solvent volume).

The amount of solvent and anti-solvent present should be enough to prepare about 0.1 molar to about 4.0 molar solutions. Typically the anti-solvent is present in an amount of 10% to 100% of the original solvent volume.

Pharmaceutically acceptable salts (or salt forms) that may be formed, other than the hydrochloride salt, can include, but are not limited to, hydrogen sulfate salt and other sulfate salts, hydrogen phosphate salt and other phosphate salts, methanesulfonate salt, p-toluenesulfonate salt, citrate salt, fumarate salt, tartrate salt, and the like. Of these, (+)-(2S,3S)-2-(3-chlorophenyl)-3,5,5-trimethyl-2-morpholinol hydrochloride salt is preferred.

Step Eight: Final recrystallization of the salt from Step Seven (7) with the objective of producing 99%+ enantiomeric excess (99% ee) pure (+)-(2S,3S)-2-(3-chlorophenyl)-3,5,5-trimethyl-2-morpholinol hydrochloride salt.

The final recrystallization is performed by conducting a “polishing” filtration and crystallization with one or more organic solvents or solvent combinations. To carry out a “final crystallization” of a drug substance to meet regulatory guidelines and regulations in the great majority of cases, it is necessary to fully dissolve the material to be crystallized, and then filter this solution. Termed a “polishing filtration”, this serves to remove extraneous material (e. g. dust, paper, cloth fibers, etc. that may be present in small amounts), prior to the final crystallization or precipitation. Accordingly, the amount of solvent present must be sufficient to dissolve all of the (+)-(2S,3S)-2-(3-chlorophenyl)-3,5,5-trimethyl-2-morpholinol hydrochloride salt. If too little solvent is used, it will not be possible to dissolve all of the hydrochloride salt and accomplish this needed procedure including the “polishing filtration purification”. Too much solvent will result in lower yields of final product (+)-(2S,3S)-2-(3-chlorophenyl)-3,5,5-trimethyl-2-morpholinol hydrochloride salt.

Suitable organic solvents include methanol, ethanol, ethyl acetate, isopropyl acetate, acetonitrile and the like or solvent mixtures thereof. The initial concentration of the (+)-(2S,3S)-2-(3-chlorophenyl)-3,5,5-trimethyl-2-morpholinol hydrochloride salt in the organic solvent or solvent mixture ranges from 0.1 molar to 4.0 molar and is capable of being filtered to remove insoluble impurities such as dust and related particulate matter.

A further invention herein is the providing of a pharmaceutical composition comprising an active ingredient of (+)-(2S,3S)-2-(3-chlorophenyl)-3,5,5-trimethyl-2-morpholinol, a pharmaceutically acceptable salt, or pharmaceutically acceptable solvate thereof prepared in accordance with the process(es) described hereinabove together with at least one pharmaceutically acceptable excipient.

The pharmaceutical composition may comprise one or more pharmaceutically acceptable carriers, diluents, and/or excipients. The carrier(s), diluent(s), and/or excipients should be acceptable in the sense of being compatible with the other ingredients of the formulation and not deleterious to the recipient thereof.

Pharmaceutical compositions may be presented in unit dose form containing a predetermined amount of active ingredient per unit dose. Such a unit may contain a therapeutically effective dose of the compound or salt or solvate of the compound, or a fraction of a therapeutically effective dose (i.e., a sub-dose), such that multiple unit dosage forms might be administered at a given time to achieve the desired therapeutically effective dose. Preferred unit dosage formulations are those containing a daily dose or sub-dose, or an appropriate fraction thereof of an active ingredient. Such pharmaceutical compositions may be prepared by any of the methods well known in the pharmacy art.

The precise therapeutically effective amount of active ingredient will depend-on a number of factors including, but not limited to, the age and weight of the subject being treated, the precise disorder requiring treatment and its severity, the nature of the formulation, and the route of administration, and will ultimately be at the discretion of the attendant physician or veterinarian. Typically, the dose given for treatment will range from about 0.001 mg/kg to about 30 mg/kg body weight of recipient (animal) per day and more usually in the rage of about 0.01 mg/kg to about 20 mg/kg body weight per day. In general, acceptable daily dosages may be from about 0.1 mg/day to about 3000 mg/day, and preferably from about 0.1 mg/kg to about 2000 mg/day. Unit doses will normally be administered once or more than once per day, preferably about 1 to about 4 times per day.

Pharmaceutical compositions may be adapted for administration by any appropriate route, for example, by oral (including buccal or sublingual), rectal, nasal, topical (including buccal, sublingual, or transdermal), vaginal or parenteral (including subcutaneous, intramuscular, intravenous or intradermal) route. Such compositions may be prepared by any method known in the art of pharmacy, for example, by bringing into association the active ingredient with the carrier(s), diluent(s), and/or excipient(s). Oral administration is most preferred.

One or more compounds prepared by the inventive process may be present with one or more non-toxic pharmaceutically acceptable ingredients and optionally, other active anti-proliferative agents, to form the pharmaceutical composition. These compositions can be prepared by applying known techniques in the art such as those taught in Remington's Pharmaceutical Sciences (Fourteenth Edition), Managing Editor, John E. Hoover, Mack Publishing Co., (1970) or Pharmaceutical Dosage Form and Drug Delivery Systems (Sixth Edition), edited by Ansel et al., publ. by Williams & Wilkins, (1995).

Depending on the route of administration, the composition can take the form of discrete units such as aerosols, creams, elixirs, emulsions, foams, whips, gels, granules, wafers, candy, inhalants, lotions, magmas, ointments, peroral solids, quick-dissolve tongue tapes (or sheets), powders, sprays, syrups, suppositories, suspensions, tablets, capsules, and tinctures. Tablets, capsules, granules, and powders are preferred. Tablets and capsules are more preferred. A once-daily tablet or capsule is most preferred. Ways of preparing these discrete units are well known in the formulation arts.

In still another embodiment of the invention there is provided a method of treatment comprising the administration (preferably oral) to a mammal of a therapeutically effective amount of an active ingredient comprising (+)-(2S,3S)-2-(3-chlorophenyl)-3,5,5-trimethyl-2-morpholinol, a pharmaceutically acceptable salt thereof, or solvate thereof prepared in accordance with the process described herein together with at least one pharmaceutically acceptable excipient. As used herein, the term “treatment” refers to alleviating the specified condition, eliminating or reducing one or more symptoms of the condition, slowing or eliminating the progression of the condition, and preventing or delaying the reoccurrence of the condition in a previously afflicted or diagnosed patient or subject. As used herein, the term “therapeutically effective amount” means an amount of the active ingredient which is sufficient, in the subject to which it is administered, to elicit the biological or medical response of a cell culture, tissue, system, animal (including human), that is being sought for instance by a physician, researcher, or clinician.

In another embodiment there is provided the use of (+)-(2S,3S)-2-(3-chlorophenyl)-3,5,5-trimethyl-2-morpholinol, a pharmaceutically acceptable salt thereof, or pharmaceutically acceptable solvate thereof prepared in accordance with process described herein in the manufacture of a medicament. Such medicament can be used for the treatment of depression (major and bipolar), attention deficit hyperactivity disorder (ADHD), anxiety, obesity, migraine, pain, sexual dysfunction in both men and women, Parkinson's disease, Alzheimer's disease, seasonal affective disorder (SAD), addiction to alcohol, addiction to cocaine, and addiction to nicotine-containing products (e.g., tobacco).

### EXAMPLES

Experiment 01a

A total of 255.8 gm (1.0 mole) of (±)-(2R*,3R*)-2-(3-chlorophenyl)-3,5,5-trimethyl-2-morpholinol is dissolved in 1.0 liter of ethanol and stirred at room temperature as enough triethylamine is added to bring the pH of this solution to about pH 11 under an inert atmosphere.

As this solution is mechanically stirred, portions of (−)-(R,R)-di-p-toluoyl-L-tartaric acid are added to the solution as well as additional triethylamine base when needed to maintain the pH of the solution at about pH 11 with stirring until the mixture becomes homogeneous. After 143.2 gm (0.5 mole) of (−)-(R,R)-di-p-toluoyl-L-tartaric acid is added and the homogeneous solution is maintained at about pH 11 for 10 minutes, additional (−)-(R,R)-di-p-toluoyl-L-tartaric acid may be added until a homogeneous solution is no longer maintained, but the pH of the solution is still between about pH 9 to about pH 11. At this point the slightly heterogeneous solution is seeded with 10 mg to 1.0 gm of (+)-(2S,3S)-2-(3-chlorophenyl)-3,5,5-trimethyl-2-morpholinol (−)-(R,R)-di-p-toluoyl-L-tartaric acid salt. The mixture is stirred for two hours to aid the precipitation (or crystallization) of the desired salt from the solution.

With continued stirring, an additional 143.2 gm (0.5 mole) of (−)-(R,R)-di-p-toluoyl-L-tartaric acid is added in portions over a 2-hour period. Triethylamine base may be added as needed to keep the pH of the solution between about pH 6.5-pH 7.5. The heterogeneous mixture is stirred overnight at room temperature. The mixture is then filtered and the solid (+)-(2S,3S)-2-(3-chlorophenyl)-3,5,5-trimethyl-2-morpholinol (−)-(R,R)-di-p-toluoyl-L-tartaric acid salt is collected by filtration and dried.

Experiment 01b

A total of 485 gm (0.90 mole) of (+)-(2S,3S)-2-(3-chlorophenyl)-3,5,5-trimethyl-2-morpholinol (−)-(R,R)-di-p-toluoyl-L-tartaric acid salt is suspended in a 1:1 mixture of 1.5 liters of ethyl acetate and 1.5 liters of deionized water and the suspension is treated with 3.0-3.5 equivalents of aqueous ammonium hydroxide to dissolve the-suspended acid salt. The organic phase is separated and the aqueous phase is extracted with 1.0 liter of ethyl acetate. The organic phase is then separated and the aqueous phase can be discarded. The organic phases are combined and washed with 1 liter of deionized water followed by 1 liter of brine. This organic phase is separated and concentrated under vacuum to yield (+)-(2S,3S)-2-(3-chlorophenyl)-3,5,5-trimethyl-2-morpholinol free base.

Experiment 01c

A total of 200 gm of (+)-(2S,3S)-2-(3-chlorophenyl)-3,5,5-trimethyl-2-morpholinol free base is dissolved in 1 liter of ethyl acetate with heating as necessary. A 5-6 molar solution of hydrochloric acid in methanol is added until a white precipitate forms and the pH of the solution reaches and is maintained at about pH 1 to about pH 2. The mixture is stirred for one hour and cooled to 10° C. with stirring. The desired (+)-(2S,3S)-2-(3-chlorophenyl)-3,5,5-trimethyl-2-morpholinol hydrochloride salt is then isolated by filtration, and the solids are washed with 49:1 ethyl acetate-methanol and are dried under vacuum. In this manner up to 99+% enantiomeric excess (99+% ee) pure quality (+)-(2S,3S)-2-(3-chlorophenyl)-3,5,5-trimethyl-2-morpholinol hydrochloride is prepared.

Experiment 01d

If required, the (+)-(2S,3S-2-(3-chlorophenyl)-3,5,5-trimethyl-2-morpholinol hydrochloride can be subjected to a polishing filtration and crystallization. This salt is dissolved in sufficient methanol to give a homogeneous solution which is filtered to remove any inert particulate matter using a sintered glass filter funnel. The filtered solution is diluted with 1-3 volumes of ethyl acetate and is concentrated under reduced pressure to selectively remove some of the methanol and to induce crystallization. The heterogeneous solution is stirred for 2-4 hours to complete the crystallization process. The solid mass of (+)-(2S,3S)-2-(3-chlorophenyl)-3,5,5-trimethyl-2-morpholinol hydrochloride salt is collected by filtration and is dried. In this manner up to 99+% enantiomeric excess (99+% ee) pure quality (+)-(2S,3S)-2-(3-chlorophenyl)-3,5,5-trimethyl-2-morpholinol hydrochloride is prepared.

Experiment 02a

A total of 255.8 gm (1.0 mole) of (±)(2R*,3R*)-2-(3-chlorophenyl)-3,5,5-trimethyl-2-morpholinol is dissolved in 1.0 liter of ethanol and is stirred at room temperature as enough triethylamine is added to bring the pH of this solution to about pH 10 under an inert atmosphere. As this solution is mechanically stirred, portions of (−)-(R,R)-di-p-toluoyl-L-tartaric acid are added to the solution as well as additional triethylamine base when needed to maintain the pH of the solution at about pH 10 with stirring until the mixture becomes homogeneous. After 143.2 gm (0.5 mole) of (−)-(R,R)-di-p-toluoyl-L-tartaric acid is added, the homogeneous solution is maintained at pH 10 for 10 minutes. Additional (−)-(R,R)-di-p-toluoyl-L-tartaric acid is added until a homogeneous solution is no longer maintained and the pH of the solution is about pH 9 to about pH 10. At this point the slightly heterogeneous solution is seeded with 10 mg to 1.0 gm of (+)-(2S,3S)-2-(3-chlorophenyl)-3,5,5-trimethyl-2-morpholinol (−)-(R,R)-di-p-toluoyl-L-tartaric acid salt. The mixture is then stirred for two hours to aid the precipitation of desired salt from the solution. With continued stirring, an additional 143.2 gm (0.5mole) of (−)-(R,R)-di-p-toluoyl-L-tartaric acid is added in portions over a two-hour period. Triethylamine base can be added as needed to keep the pH of the solution between about pH 6.5-pH 7.5. The heterogeneous mixture is then stirred overnight at room temperature. The mixture is filtered and the solid (+)-(2S,3S)-2-(3-chlorophenyl)-3,5,5-trimethyl-2-morpholinol (−)-(R,R)-di-p-toluoyl-L-tartaric acid salt is then collected by filtration and is dried.

Experiment 02b

A total of 485 gm (0.90 mole) of (+)-(2S,3S)-2-(3-chlorophenyl)-3,5,5-trimethyl-2-morpholinol (−)-(R,R)-di-p-toluoyl-L-tartaric acid salt is suspended in a 1:1 mixture of 1.5 liters of ethyl acetate and 1.5 liters of deionized water. The suspension is treated with about 3.0-3.5 equivalents of aqueous ammonium hydroxide to dissolve the suspended salt. The organic phase is separated and the aqueous phase is extracted with 1.0 liter of ethyl acetate. The organic phase is separated and the aqueous phase can be discarded. The organic phases are combined and washed with 1 liter of deionized water followed by 1 liter of brine. This organic phase is separated and concentrated under vacuum to yield crude (+)-(2S,3S)-2-(3-chlorophenyl)-3,5,5-trimethyl-2-morpholinol free base.

Experiment 02c

A total of 200 gm of crude (+)-(2S,3S)-2-(3-chlorophenyl)-3,5,5-trimethyl-2-morpholinol free base is dissolved in 1 liter of ethyl acetate with heating as necessary. A 5-6 molar solution of hydrochloric acid in methanol is added until a white precipitate forms and the pH of the solution reaches and is maintained at about pH 1 to about pH 2. The mixture is stirred for one hour and is then cooled to 10° C. with stirring. The desired (+)-(2S,3S)-2-(3-chlorophenyl)-3,5,5-trimethyl-2-morpholinol hydrochloride salt is isolated by filtration, and the solids are washed with 49:1 ethyl acetate-methanol and dried under vacuum. In this manner up to 99+% enantiomeric excess (99+% ee) pure quality (+)-(2S,3S)-2-(3-chlorophenyl)-3,5,5-trimethyl-2-morpholinol hydrochloride is prepared.

Experiment 02d

If required, the (+)-(2S,3S)-2-(3-chlorophenyl)-3,5,5-trimethyl-2-morpholinol hydrochloride can be subjected to a polishing filtration and crystallization. This salt is dissolved in sufficient methanol to give a homogeneous solution which is filtered to remove any inert particulate matter using a sintered glass filter funnel. The filtered solution is diluted with 1-3 volumes of ethyl acetate and concentrated under reduced pressure to selectively remove some of the methanol and to induce crystallization. The heterogeneous solution is stirred for 2-4 hours to complete the crystallization process and the (+)-(2S,3S)-2-(3-chlorophenyl)-3,5,5-trimethyl-2-morpholinol hydrochloride salt is collected by filtration and dried. In this manner up to 99+% enantiomeric excess (99+% ee) pure quality (+)-(2S,3S)-2-(3-chlorophenyl)-3,5,5-trimethyl-2-morpholinol hydrochloride is prepared.

All cited patents, publications, co-pending applications, and provisional applications referred to in this application are herein incorporated by reference.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A process for preparing (+)-(2S,3S)-2-(3-chlorophenyl)-3,5,5-trimethyl-2-morpholinol in its free base, its salt form, or both, which process comprises a dynamic kinetic resolution by equilibrating the two chiral centers of (±)-(2R*,3R*)-2-(3-chlorophenyl)-3,5,5-trimethyl-2-morpholinol.

2. The process according to claim 1 which process comprises the steps of:
(1) treating (±)-(2R*,3R*)-2-(3-chlorophenyl)-3,5,5-trimethyl-2-morpholinol dissolved in a solvent with at least one base to give a solution having a pH of about pH 8 to a pH of about pH 12;
(2) adding at least 0.5 equivalent of a chiral acid with stirring while maintaining the pH of the solution between about pH 8 to about pH 12 with additional base;
(3) adding seed crystals of the desired chiral acid salt of (+)-(2S,3S)-2-(3-chlorophenyl)-3,5,5-trimethyl-2-morpholinol with stirring; and
(4) adding at least 0.5 equivalent of additional chiral acid at pH 8 to pH 12, with additional base added as needed, and then adjusting the pH of the mixture until the pH reaches about pH 6 to pH 8 with acid.

3. The process according to claim 2, wherein the chiral acid is a protected or unprotected chiral tartaric acid.

4. The process according to claim 2, wherein the chiral acid is selected from the group consisting of L-tartaric acid, D-tartaric acid, (−)-(R,R)-di-p-benzoyl-L-tartaric acid, (+)-(S,S)-di-p-benzoyl-D-tartaric acid, (−)-(R,R)-di-p-toluoyl-L-tartaric acid, (+)-(S,S)-di-p-toluoyl-D-tartaric acid, and (−)-(1R)-10-camphorsulfonic acid, (+)-(1S)-10-camphorsulfonic acid D-malic acid, L-malic acid, D-mandelic acid or L-mandelic acid.

5. The process according to claim 4, wherein the chiral acid is (−)-(R,R)-di-p-toluoyl-L-tartaric acid.

6. The process according to claim 2, wherein the chiral acid is a substituted or unsubstituted chiral camphorsulphonic acid.

7. The process according to claim 6, wherein the chiral camphorsulphonic acid is selected from the group consisting of (−)-(1R)-10-camphorsulphonic acid and (+)-(1S)-10-camphorsulfonic acid.

8. The process according to claim 2, wherein the base is an inorganic base or an organic base.

9. The process according to claim 8, wherein the inorganic base is at least one selected from the group consisting of alkali metal hydrogen carbonates, alkali metal carbonates, alkali metal hydroxides, and ammonium hydroxide.

10. The process of claim 9 wherein said alkali metal hydrogen carbonates are selected from the group consisting of sodium hydrogen carbonate and potassium hydrogen carbonate; and wherein the alkali metal carbonates are selected from the group consisting of sodium carbonate and potassium carbonate.

11. The process according to claim 8, wherein the organic base is at least one selected from the group consisting of aliphatic amine bases and aromatic amine bases.

12. The process according to claim 11, wherein the organic base is triethylamine.

13. The process according to claim 2, wherein the solvent is a protic solvent.

14. The process according to claim 13, wherein said protic solvent is at least one selected from the group consisting methanol and ethanol.

15. The process according to claim 2, wherein the seed crystal is a (+)-(2S,3S)-2-(3-chlorophenyl)-3,5,5-trimethyl-2-morpholinol (−)-(R,R)-di-p-toluoyl-L-tartaric acid salt.

16. The process according to claim 2, wherein said at least 1.0 equivalent of a chiral acid is added slowly over a period of about 1 to about 4 hours.

17. The process according to claim 2, wherein said additional chiral acid is added slowly over a period of about 1 to about 4 hours.

18. The process according to claim 2, wherein said solution or mixture of step (1) has a pH of about pH 9 to about pH 11.

19. The process according to claim 2, wherein said solution or mixture of step (2) has a pH of about pH 9 to about pH 11.

20. The process according to claim 2, wherein said solution or mixture of step (4) has a final pH of about pH 6.5 to about pH 7.5.

21. The process according to claim 2, further comprising the step of:
(5) isolating a solid acid salt of (+)-(2S,3S)-2-(3-chlorophenyl)-3,5,5-trimethyl-2-morpholinol from said solution or mixture of step (4).

22. The process according to claim 21, wherein said isolation is performed using a sintered glass filter funnel, a Gooch filter, a pan filter, and a Rosemund filter.

23. The process according to claim 21, further comprising the step of:
(6) converting said solid acid salt of (+)-(2S,3S)-2-(3-chlorophenyl)-3,5,5-trimethyl-2-morpholinol of step (5) into (+)-(2S,3S)-2-(3-chlorophenyl)-3,5,5-trimethyl-2-morpholinol free base.

24. The process according to claim 23, wherein said solid acid salt is converted into its free base form of the acid salt with a base.

25. The process according to claim 24, wherein said base is a strong base in an excess amount.

26. The process according to claim 25, wherein said base is selected from the group consisting of ammonium hydroxide, potassium hydroxide, sodium hydroxide, and mixtures thereof in water.

27. The process according to claim 23, further comprising the step of:
(7) converting the (+)-(2S,3S)-2-(3-chlorophenyl)-3,5,5-trimethyl-2-morpholinol free base of step (6) into a (+)-(2S,3S)-2-(3-chlorophenyl)-3,5,5-trimethyl-2-morpholinol acid salt.

28. The process according to claim 27, wherein said free base is converted into said hydrochloride salt by addition of more than one equivalent of hydrochloric acid and a anti-solvent or by addition of more than one equivalent of hydrogen chloride gas and a anti-solvent, such that the pH of the solution or mixture reaches a pH of about pH 1 to a pH of about pH 2.

29. The process according to claim 28, wherein said solvent is selected from the group consisting of methanol, ethanol, ethyl acetate, isopropyl acetate, acetonitrile, and mixtures thereof; and said anti-solvent is selected from the group consisting of ethyl acetate, isopropyl acetate, ethyl ether, methyl t-butyl ether, and mixtures thereof.

30. The process according to claim 28, further comprising the step of:
(8) recrystallizing the (+)-(2S,3S)-2-(3-chlorophenyl)-3,5,5-trimethyl-2-morpholinol hydrochloride salt of step (7).

31. The process according to claim 30, wherein said recrystallization of step (8) is performed by polishing filtration and crystallization with one or more organic solvents.

32. The process according to claim 31, wherein said one or more organic solvent is selected from the group consisting of methanol, ethanol, ethyl acetate, isopropyl acetate, and acetonitrile.

33. The process according to claim 27 wherein said acid salt form is selected from the group consisting of
(i) (+)-(2S,3S)-2-(3-chlorophenyl)-3,5,5-trimethyl-2-morpholinol hydrochloride salt;
(ii) (+)-(2S,3S)-2-(3-chlorophenyl)-3,5,5-trimethyl-2-morpholinol hydrogen sulfate salt;
(iii) (+)-(2S,3S)-2-(3-chlorophenyl)-3,5,5-trimethyl-2-morpholinol hydrogen phosphate salt;
(iv) (+)-(2S,3S)-2-(3-chlorophenyl)-3,5,5-trimethyl-2-morpholinol methanesulfonate salt;
(v) (+)-(2S,3S)-2-(3-chlorophenyl)-3,5,5-trimethyl-2-morpholinol p-toluenesulfonate salt;
(vi) (+)-(2S,3S)-2-(3-chlorophenyl)-3,5,5-trimethyl-2-morpholinol citrate salt;
(vii) (+)-(2S,3S)-2-(3-chlorophenyl)-3,5,5-trimethyl-2-morpholinol fumarate salt; and
(viii) (+)-(2S,3S)-2-(3-chlorophenyl)-3,5,5-trimethyl-2-morpholinol tartrate salt.

34. A pharmaceutical composition comprising an active ingredient of (+)-(2S,3S)-2-(3-chlorophenyl)-3,5,5-trimethyl-2-morpholinol, a pharmaceutically acceptable salt thereof, or pharmaceutically acceptable solvate thereof prepared in accordance with claim 1 together with at least one pharmaceutically acceptable excipient.

35. A method for the treatment of depression, attention deficit hyperactivity disorder, anxiety, obesity, migraine, pain, sexual dysfunction, Parkinson's disease, Alzheimer's disease, seasonal affective disorder, addiction to alcohol, addiction to cocaine, or addiction to nicotine-containing products comprising the oral administration to a mammal of an active ingredient comprising (+)-(2S,3S)-2-(3-chlorophenyl)-3,5,5-trimethyl-2-morpholinol, a pharmaceutically acceptable salt thereof, or a pharmaceutically acceptable solvate thereof prepared in accordance with claim 1 together with at least one pharmaceutically acceptable excipient.

36. (canceled)
